(19) United States
(12) Patent Application Publication
(10) Pub. No.: US 2003/0152481 A1
(43) Pub. Date: Aug. 14, 2003

(54) Process for decontaminating an enclosed space

(76) Inventor: Walter Birnecker, Eichgraben (AT)

Correspondence Address: LERNER AND GREENBERG, P.A., POST OFFICE BOX 2480, HOLLYWOOD, FL, 33022-2480, US

(21) Appl. No.: 10/075,581

(22) Filed: Feb. 14, 2002

(51) Int. Cl. A61L 2/16; A61L 2/00

(52) U.S. Cl. 422/28; 422/1

(57) ABSTRACT

Decontamination is disclosed of an enclosed space, which can be at least one cubic meter, by liquid particles of controlled dimensions containing a contaminant neutralizing agent and able to reach all parts of the space and remain suspended therein for hours. The particles are generated by passing a carrier gas and a liquid contaminant neutralizing agent having controlled viscosity and surface tension through a Venturi generator. Chemical and microbiological contaminants are decontaminated according to the invention.

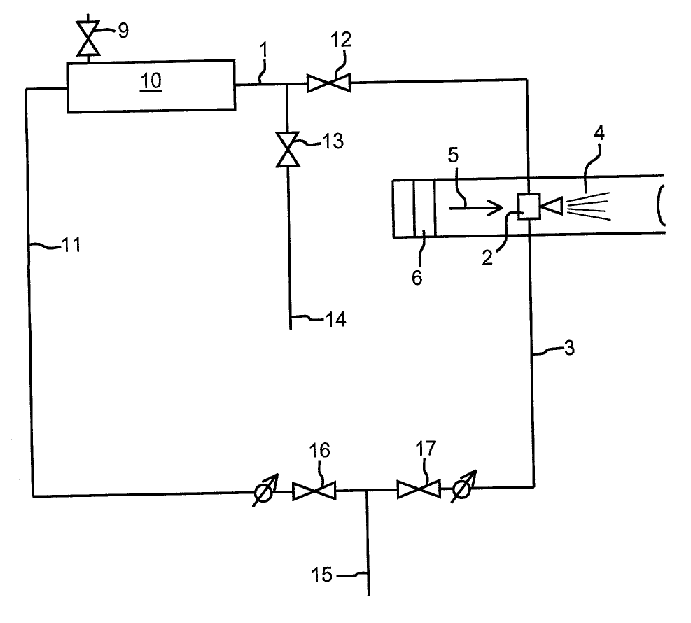

### BACKGROUND OF THE INVENTION

### FIELD OF THE INVENTION

This invention relates to the decontamination of enclosed spaces, the surfaces defining these spaces, and the surfaces of objects within these spaces by removing therefrom chemical or microbiological contaminants such as air pollutants, pesticides, drugs of abuse, bacteria, fungi, and viruses.

Each year, especially during the winter months, many thousands of people suffer from infections commonly known as grippe, flu, and influenza. The infective microorganisms believed to be responsible pass from an infected person to others, particularly in crowded locations.

As a public health measure, it is therefore considered desirable to reduce the number of infective microorganisms in enclosed spaces. p Fumigation to diminish the number of infective microorganisms in enclosed spaces intended to be used by a large number of people or inhabited by farm animals has long been practiced. A variety of gaseous and liquid agents have been used, including inorganics such as chlorine, chlorine dioxide, hydrogen peroxide, iodine, ozone, and permanganate salts, and numerous organic antimicrobials.

Chemical contaminants have been mitigated by various techniques adapted to the particular contaminant, including electrostatic precipitation, mechanical displacement by high-pressure steam and other gases under pressure, neutralization by appropriate reagents, and washing of surfaces with any of a host of cleansing formulations.

All of these methods are subject to significant physical limitations. Gaseous treating agents are subject to the difficulty of providing sufficient mass of active agent to be effective, since in any given volume the mass of gaseous agent cannot be greater than that determined by the ideal gas law PV=nRT, P being pressure, V volume, n the number of moles i.e. the mass, R the gas constant, and T the temperature in K. Condensed phase agents such as liquids, on the other hand, must overcome gravity to reach all parts of an enclosed space including obstructed areas before eventually settling to the lowest level.

Individual disclosures in this field include the following:

Lembke, U.S. Pat. No. 6,224,827 B1 of May 1, 2001 disclosed a water-free alcohol disinfectant capable of destroying spores, preferably including 0.5-3% by weight hydrogen peroxide for use in industrial context for bacteriological cleaning of surfaces.

Belfer, U.S. Pat. No. 6,106,854 of Aug. 22, 2000 disclosed an asepsis disinfectant composition in liquid form including an anti-infective germicidal and antiseptic agent selected from the group consisting of hydrogen peroxide, urea hydrogen peroxide and betaine hydrochloride; a sanitizer and bactericide selected from the group consisting of iodine, iodine pentafluoride, iodine monobromide, iodine chloride, iodine halides, iodophors and tetraglycine hydroperiodide; a cleansing agent, an antioxidant and stabilizing agent, a pH adjuster, and a diluent. The composition may also include a gas propellant for acting as a carrier in order to provide a pressurized aerosol spray.

Childers. U.S. Pat. No. 5,906,794 of May 25, 1999 disclosed a continuous operation closed loop system for conducting closed loop flow through vapor phase decontamination. In the optimized method, a flow of carrier gas is recirculated in a closed loop conduit circuit that leads into, through, and out of asealble chamber. A liquid decontaminant is vaporized and delivered into the carrier gas flow entering the chamber, and then converted to a form suitable for disposal after exiting the chamber. The liquid decontaminant preferably comprises aqueous hydrogen peroxide. The system includes a liquid sterilant vaporizer unit for delivering a vaporized liquid sterilant into the carrier gas. Liquid sterilant is preferably atomized in an atomizer fluidly connected to the vaporizer, and is delivered to the vaporizer in the form of a fine mist to increase the likelihood of complete vaporization.

Childers, U.S. Pat. No. 5,492,672 of Feb. 20, 1996 disclosed a method for sterilizing items using a multi-component vapor phase sterilant where one component is water. The method includes injecting sterilant vapor into a chamber at subatmospheric pressure controlled at various levels at various times, discontinuing the flow of sterilant vapor into, through, and out of the chamber to hold the sterilant vapor therein for a period of time sufficient to permit the sterilant vapor to permeate the chamber and come into effective contact with items to be sterilized, and repeating the steps of flowing sterilant and discontinuing the flow of sterilant in alternating fashion until sterilization of goods in the chamber is achieved. The sterilizer in which the method is practiced incluldes a sterilization chamber, a source of liquid sterilant, a vacuum pump , a vaporization chamber upstream from the sterilization chamber, and a system of valves. The pump draws the liquid sterilant from its source through a filter and on to the inlet of the vaporization chamber. Air is drawn through a filter and into the inlet of the vaporization chamber. A flow restrictor (venturi) is provided in-line, upstream of the inlet so that the air or the air and sterilant mixture passes through the flow restrictor to the inlet of the vaporization chamber.

Hool, U.S. Pat. No. 3,982,022 of Sep. 21, 1976 disclosed a composition for the control of microorganisms containing a combination of active substances consisting of a first compound halogen substituted 2-phenoxyphenol and a second compound of the formula

(X)ₙ—C₆H₅₋ₙ—Y—CH₂—OH

wherein n represents 0 or a whole number from 1 to 5, X represents a halogen atom, and Y represents a radical of the formula —O—CH₂— or —CH₂— or the direct bond, together with the usual carriers and/or dispersing agents.

Mullen, U.S. Pat. No. 3,635,836 of Jan. 18, 1972 disclosed thickened viscous or gelled acid dispersions comprising a selected protic acid and particulate proteinaceous material derived from legume seeds or cottonseeds. The dispersions are useful for applying strongly acid films to surfaces of metal, stone, ceramic, textile or wooden articles for the purpose of treating the surface. Once the treatment has been completed the dispersions can be washed off. The composition of the thickened dispersions comprises by weight of the total composition about 1.5 to 20.0 percent particulate proteinaceous material, about 20.0 to 55.0 percent protic acid, and about 40.0 to 75.0 percent water, alkanol, or alkylcarbonyl compounds. Useful alkanols are mono- and polyhydroxy alcohols preferably containing 10 or fewer carbon atoms, and their cyclic or branched counterparts such as cyclohexanol.

### SUMMARY OF THE INVENTION

It is accordingly an object of the invention to provide a process for decontaminating an enclosed space that overcomes the above-mentioned disadvantages of the prior art devices and methods of this general type, in which a contaminated enclosed space as well as the surfaces defining the space and the surfaces of any objects within the space is decontaminated by treatment with liquid particles of controlled dimensions containing a contaminant neutralizing agent and able to reach all parts of the space and remain suspended therein for hours.

It is a further object of the invention to return the treated enclosed space to access and practical use in a simple manner. With the foregoing and other objects in view, there is provided, in accordance with the invention, a process for decontaminating an enclosed space containing a contaminant and surfaces surrounding the space, which comprises the steps of

providing a liquid contaminant neutralizing agent having a predetermined surface tension and viscosity,

providing a source of carrier gas under a first pressure,

providing a source of carrier gas under a second pressure greater than the first pressure,

injecting the liquid contaminant neutralizing agent into a nozzle with carrier gas under the first pressure, thereby generating a loaded stream of contaminant neutralizing agent and carrier gas,

injecting a stream of carrier gas under the second pressure and the loaded stream into a venturi generator nozzle, thereby mixing the streams and generating a stream comprising carrier gas and particles of contaminant neutralizing agent having a predetermined particle size distribution,

injecting the stream comprising carrier gas and the particles of contaminant neutralizing agent into the enclosed space,

causing particles of contaminant neutralizing agent to dwell in the enclosed space for a predetermined time, thereby decontaminating the enclosed space,

and removing particles of contaminant neutralizing agent from the treated enclosed space.

With the foregoing and other objects in view, there is also provided, in accordance with the invention, a process as above which further comprises reducing the flow of the loaded stream subsequent to the dwell time of contaminant neutralizing agent in the enclosed space. The flow of the loaded stream can be reduced to a slower rate or to a rate of zero, so that the flow of loaded stream is stopped entirely.

The process of the invention can be followed by injection of pure carrier gas into the enclosed space subsequent to the dwell time of contaminant neutralizing agent therein. Such injection of pure carrier gas serves to displace from the enclosed space the spent contaminant neutralizing agent solution and conversion products resulting from its use, thereby facilitating access to and use of the decontaminated enclosed space.

It is a feature of the invention that particles of contaminant neutralizing agent generated according to the invention can remain suspended in the enclosed space for at least one hour. This assures effective contact of the contaminant neutralizing agent with contaminant to be treated at any place throughout the enclosed space, including surfaces defining the space as well as objects located therein. As a result, irregularly shaped spaces as well as spaces including irregularly shaped objects are effectively treated. As a further result, the process can be applied to relatively large spaces having volumes of at least one cubic meter and ranging up to several thousand cubic meters.

The predetermined viscosity of the liquid contaminant neutralizing agent according to the invention is in the range from 0.15 to 1500 centipoises measured at 20° C., preferably from 0.164 to 1499 centipoises so measured.

The predetermined surface tension of the liquid contaminant neutralizing agent according to the invention is in the range from 10 to 100 dyn/cm measured against air or liquid vapor at 20° C., preferably from 17.0 to 72.5 dyn/cm so measured.

The predetermined particle size distribution according to the invention is such that at least 90% of the particles of liquid contaminant neutralizing agent are in the range from 1 micron (μm) to 100 microns in diameter in a Gaussian distribution, and preferably at least 95% of the particles of liquid contaminant neutralizing agent are in the range from 8 micron to 55 micron according to Gaussian distribution.

The liquid contaminant neutralizing agent according to the invention can be a liquid substance having the desired effectiveness in neutralizing a contaminant as well as the predetermined properties of viscosity and surface tension. Usually, however, the liquid contaminant neutralizing agent according to the invention is a solution comprising a substance having the desired effectiveness in neutralizing a contaminant and a solvent, formulated so as to provide the predetermined viscosity and surface tension.

According to a further feature of the invention, an outlet leading to a drain can be provided upstream of the venturi generator for the removal of oversize particles from the loaded stream prior to injection into the venturi generator.

Other features which are considered as characteristic for the invention are set forth in the appended claims.

Although the invention is illustrated and described herein as embodied as a process for decontaminating an enclosed space, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawing.

Other features which are considered as characteristic for the invention are set forth in the appended claims.

Although the invention is illustrated and described herein as embodied in a process for decontaminating an enclosed space, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE DRAWING

The single FIGURE is a circuit loop diagram illustrating the flow of contaminant neutralizing agent solution and carrier gas through a venturi generator in which particles of predetermined particle size distribution are generated and injected into the enclosed space to be decontaminated.

### DESCRIPTION OF THE PREFERRED EMBODIMENTS

The liquid contaminant neutralizing agent can comprise any substance effective in neutralizing a contaminant of concern. The agent is selected primarily for maximal effectiveness and secondarily for the ability to avoid or minimize undesirable effects such as toxicity to humans, corrosivity to the surfaces of the space to be decontaminated, difficulties in removing the agent from the treated space after use, and pollution of the environment after removal from the treated space.

Subject to these considerations, non-limiting neutralizing agents for contaminants having acid properties and contaminants readily hydrolyzed to acidic substances are alkalies such as borax, ethanolamine, potassium bicarbonate, potassium carbonate, potassium hydroxide, sodium carbonate, sodium hydroxide, tetrapotassium pyrophosphate, triethanolamine and trisodium phosphate. Especially potassium hydroxide: water: ethanol=1:1:1. Such alkalies are effective neutralizing agents for acidic contaminants such as phosgene, diisopropyl phosphorofluoridate and certain halogenated pesticides.

Similarly, neutralizing agents for other chemical contaminants are selected on the basis of known principles of chemical (reactivity and solubilization.

Subject to the above considerations, non-limiting neutralizing agents for microbiological contaminants such as live bacteria, bacterial spores, fungi and viruses can be any agent known to be effective against the organism of concern. A plurality of effective agents can be used, especially for purposes of preventive prophylaxis.

Preferred contaminant neutralizing agents include organic halogen compounds such as hexachlorocyclohexane and aldehydes such as formaldehyde, 2-ethylhexan-1-al, and pentane-1,5-dial.

A particularly preferred contaminant neutralizing agent for microbiological contaminants is 2-phenylethanol. This alcohol unites high antimicrobial effectiveness with low toxicity (oral LD₅₀ in rats=1790 mg/kg), agreeable odor properties and substantial absence of side effects.

When the contaminant neutralizing agent comprises a solvent, the solvent can be aqueous or non-aqueous, as required by the solubility properties of the substance having the desired effectiveness in neutralilzing a contaminant. Where practical the solvent comprises water. Frequently the solvent also comprises a water soluble organic liquid to enhance the solubility of the effective substance. Suitable water soluble organic liquids able to solubilize many effective substances include acetone; aliphatic alcohols having 1 to 4 carbon atoms such as methanol, ethanol, 1-propanol, 2-propanol, 1-butanol, 2-butanol, 2-methyl-1-propanol and 2-methyl-2-propanol; dimethyl sulfoxide; and ether alcohols having 1-2 ether groups and 1-2 hydroxyl groups such as diethylene glycol, 2-methoxyethanol, 2-ethoxyethanol, and 2-(2′-butoxyethoxy)ethanol. Mixtures of such liquids can be used if desired, with the relative proportions of the components such that solubility in the mixture of the effective substance and water if present is assured.

The effective substance can be present in the liquid contaminant neutralizing agent in any convenient concentration consistent with the predetermined surface tension and viscosity. Typical concentrations of effective substance are in the range from 0.5% to 20% by weight, preferably 1% to 10% by weight.

The surface tension of the liquid contaminant neutralizing agent is primarily a function of the nature and concentration of the effective substance and the solvent. When necessary, a surfactant is included to diminish the surface tension. The nature of the surfactant is not critical. Anionic, cationic, nonionic, and zwitterionic surfactants can be used. Within each of these categories a plentiful selection is commercially available. For a convenient compilation of surfactants within each of the named categories reference can be had to Trinh et al., U.S. Pat. No. 5,540,853, column 7 line 5 to column 14 line 4, here incorporated by reference.

As is well known in the art, surfactants vary in their tendency to cause foaming. Low foam surfactants are available and are particularly preferred.

The viscosity of the liquid contaminant neutralizing agent is primarily a function of the nature and concentration of the effective substance and the solvent and may be influenced by surfactant when present. These can be judiciously selected to achieve the predetermined viscosity. Secondary adjustments of viscosity can be accomplished by modest additions of organic liquids having a large viscosity depressing effect per unit weight; a preferred viscosity depressant is 1-butanol.

The nature of the carrier gas is not critical. Any non-corrosive gas can be used. Air is preferred for reasons of convenience and cost.

Referring now to the FIGURE of the drawing, a loaded stream of liquid contaminant neutralizing agent under gas with a first pressure is conveyed into a venturi generator, nozzle or flow restrictor 2 and is there mixed with a stream 3 of carrier gas under a second pressure greater than the first pressure. The interaction of the streams 1 and 3 in the flow restrictor 2 results in great turbulence, intensive mixing, and expulsion of a stream of particles 4 of liquid contaminant neutralizing agent having a predetermined particle size distribution through a connecting line into the enclosed space to be treated (not shown).

If desired, the stream of particles 4 can be accompanied or followed by a gas stream, preferably a stream of air, 5, issuing from an optional variable speed blower 6. The blower speed is regulated as a function of the power supply to help in adjusting the concentration of liquid contaminant neutralizing agent in the stream of particles 4.

The loaded stream 1 is generated by mixing liquid contaminant neutralizing agent from a storage container 10 supplied from a feed vessel 9 with carrier gas under a first pressure 11, and passes through a valve 12 to the flow restrictor 2. Deposition of oversize particles only takes place on the two rotating plates 13, 14 for the purpose of draining the remaining liquid.

Carrier gas is delivered from a pressurized gas supply point 15. Multiple pressurized gas supply points can be used if available. From a single pressurized gas supply point, a stream of gas is split by passing through two pressure regulator valves 16 and 17 to provide stream 11 at a first pressure and stream 3 at a second pressure greater than the first pressure. The stream 11 of carrier gas at the first pressure pumps the liquid contaminant neutralizing agent before passing through valve 12 to the nozzle 2 , while the stream 3 of carrier gas at the second pressure passes directly into the flow restrictor 2.

The first pressure is preferably in the range of 0.02 to 0.5 bar. The second, greater pressure, is less than or equal to 10 bar, preferably in the range of 1 to 10 bar.

It can be seen that the process of the invention provides several parameters that can be set so as to achieve a narrow particle size distribution of contaminant neutralizing agent in the enclosed space able to remain suspended and in contact with all interior surfaces so as to accomplish effective neutralization of contaminant therein. These parameters are the viscosity and surface tension of the liquid contaminant neutralizing agent, the gas pressure at the constriction of the venturi generator, the carrier gas flow rate and the configuration of the venturi generator. Since each of these parameters is amenable to independent control, a great variety of settings is available to enable effective neutralization of contaminants in the enclosed space to be treated.

The flow restriction applied by the venturi generator can be constant as a result of the construction of the device, or can vary with time as a result of the operation of moving parts therein. An example of venturi generator with moving parts includes two circular disks with holes or slots in parallel planes rotating at different speeds. As the disks rotate, both in the same direction or in opposite directions, the incoming stream is constricted to varying extent as the openings in the disks are alternately reinforced and blocked.

A venturi generator suitable for the practice of this invention is commercially available as “Venturigenerator Typ VII” from Chemlab GmbH, A-3032 Eichgraben, Austria.

The following Examples are provided to further illustrate the operation of the process of the invention without limiting its scope, which is defined by the appended claims.

### EXAMPLE 1

### Disinfection of a School Ventilating Unit with Built-in Humidification

The purpose of this example was the removal of streptococci and pneumococci pathogenic to humans and removal of legionellae if present.

The volume of the ventilating unit was approximately 120 m³

Apparatus settings

Carrier air speed 20 m³/min.

Venturi ratio 1:940

Feed rate of the disinfectant solution 240 ml/min.

Properties of the disinfectant solution:

Viscosity: 48·10⁻³Pa·s (48 centipoises) adjusted with n-Butanol

Surface tension: 22.5 dyn/cm adjusted with 10% DBS (dodecylbenzenesulfonic acid) Na salt in distilled water.

Disinfectant composition: 58.5% by weight distilled water

40% wt. 1-propanol

1.5% 2-phenylethanol

The progress of disinfection was followed by determining the total number of germs as the average of 12 individual measurements.

Procedure

The venturi generator was connected to a view opening in the ventilating unit, normally kept closed except when used for cleaning, using a flexible hose of ID 100 mm.

The total number of microorganisms measured before treatment was greater than 10⁶ per square centimetre, including such species as Streptococcus aureus, Aspergillus niger, Geotrichum candidum, Penicilliuim commune and Candida albicans.

Precisely controlled venturi particles of disinfectant were blown in for 10 minutes while maintaining the specified apparatus parameter.

The disinfectant particles were allowed to act for one hour without any air blow, followed by 10 minutes of blowing ordinary air through the treated space.

Result of treatment.

After treatment no microorganism growth was detected. Specifically, no Legionellae were detected.

As a test for possible activity against Anthrax, the treatment was repeated with a special disinfecting solution and test culture of cereus subtilis inserted into the ventilating unit, measuring 147-238 microorganisms per plate. After the treatment, a total of 1-3 microorganisms per plate was found.

Thus the total number of germs was reduced by 99.2% (average of 12 experiments).

### EXAMPLE 2

### Disinfection of a Stable in a Swine Feeding Establishment

Purpose: decontamination of pathogenic viruses microorganisms.

Volume of the stable approximately 7000 m³

Carrier air speed 250 m³/min.

Venturi ratio 1:920

Feed rate of the disinfectant solution 420 ml/min.

Properties of the disinfectant solution:

Viscosity: 42·10⁻³Pa·s (42 centipoises) adjusted with n-Butanol

Surface tension: 22.7 dyn/cm adjusted with 10% DBS (dodecylbenzenesulfonic acid) Na salt in distilled water.

Disinfectant composition: Chemlab Clean Air Type XI—a disinfectant commercially available from Chemlab GmbH, A-3032 Eichgraben, Austria.

Procedure

The venturi generator was connected to an air vent in the stable through a flexible hose of ID 100 mm. All animals were removed from the stable and the doors and windows closed.

The total number of microorganisms measured before treatment was greater than 10⁶ per square centimetre, including microorganisms like Aspergillus niger, Candida albicans, Clostridium tetani, Corynebacterium renale, and Nocardia asteroides.

Precisely controlled venturi particles of disinfectant were blown in for 1 hour while maintaining the specified apparatus parameters.

The disinfectant particles were allowed to act for one hour without any air blow, followed by 2 hours of blowing ordinary air through the treated space with one door open.

Result

No pathogenic microorganisms could be detected at 20 measuring points in the stable.

In a control test with cereus subtilis and a special disinfection liquid , ten test cultures were distributed throughout the space, diluted to 121-209 microorganisms per plate. After treatment, 1-3 microorganisms per plate were found, representing 99.3% reduction (average of 10 experiments) of the total number.

It should be noted that cereus subtilis is a very resistant anaerobic microorganism used having closely related properties to Anthrax (bacillae anthracis) where safety considerations prohibit experimentation with the latter.

Additional applications of the process of the invention include surface treatments such as the removal of rust particles from stainless steel structures such as long pipes and conduits, removal of fats and other organic air contaminants from air in kitchens and eating places, as well as removal of leaked and spilled chemical contaminants in workplaces.

I claim:

1. A process for decontaminating an enclosed space containing a contaminant and surfaces surrounding the space, comprising the steps of
providing a liquid contaminant neutralizing agent having a predetermined surface tension and viscosity,
providing a source of carrier gas under a first pressure,
providing a source of carrier gas under a second pressure greater than the first pressure,
injecting said neutralizing agent into a stream of carrier gas under the first pressure, thereby generating a loaded stream of contaminant neutralizing agent and carrier gas,
injecting a stream of carrier gas under the second pressure and the loaded stream into a venturi generator nozzle, thereby mixing the streams and generating a stream comprising carrier gas and particles of contaminant neutralizing agent having a predetermined particle size distribution,
injecting the stream comprising carrier gas and particles of contaminant neutralizing agent into the enclosed space,
causing particles of contaminant neutralizing agent to dwell in the enclosed space for a predetermined time, thereby decontaminating the enclosed space,
and removing particles of contaminant neutralizing agent from the treated enclosed space.

2. The process of claim 1, wherein the liquid contaminant neutralizing agent comprises a solvent.

3. The process of claim 1, wherein the carrier gas is air.

4. The process of claim 1, wherein the contaminant neutralizing agent comprises an antimicrobial.

5. The process of claim 4, wherein the antimicrobial is 2-phenylethanol.

6. The process of claim 1, wherein the contaminant neutralizing agent comprises an alkali.

7. The process of claim 1, wherein the first pressure is 0.02-0.5 bar.

8. The process of claim 1, wherein the second pressure is 1-10 bar.

9. The process of claim 1, wherein the volume of the enclosed space is at least 1 m³.

10. The process of claim 1, further comprising reducing the flow of the loaded stream subsequent to the dwell time of contaminant neutralizing agent in the enclosed space.

11. The process of claim 10, wherein the flow of the loaded stream is stopped.

12. The process of claim 1, further comprising injection of pure carrier gas into the enclosed space subsequent to the dwell time of contaminant neutralizing agent.

13. The process of claim 1, wherein particles of contaminant neutralizing agent remain suspended in the enclosed space for at least one hour.

14. The process of claim 1, wherein the viscosity of the contaminant neutralizing agent solution, measured at 20° C., is in the range from 0.15 to 1500 centipoises.

15. The process of claim 1, wherein the surface tension of the contaminant neutralizing agent solution, measured at 20° C., is in the range from 15 to 75 dyn/cm.

16. The process of claim 1, wherein at least 90% of the particles of contaminant neutralizing solution are in the range from 1 to 100 microns.

17. The process of claim 2, wherein the solvent comprises water and 1-propanol.

18. The process of claim 1, wherein the decontamination solution is potassium hydroxide:water:ethanol=1:1:1.
